(19) United States
(12) Patent Application Publication
(10) Pub. No.: US 2008/0004491 A1
(43) Pub. Date: Jan. 3, 2008

(54) METHOD FOR FABRICATING OPTICAL FIBER

(75) Inventor: Satoshi KARASAWA, Saitama (JP)

Correspondence Address: GREENBLUM & BERNSTEIN, P.L.C., 1950 ROLAND CLARKE PLACE, RESTON, VA, 20191, US

(73) Assignees: UNIVERSITY OF WASHINGTON, Seattle, WA (US); PENTAX CORPORATION, Tokyo (JP)

(21) Appl. No.: 11/427,219

(22) Filed: Jun. 28, 2006

(51) Int. Cl. B29D 11/00; C03C 15/00; A61B 1/00

(52) U.S. Cl. 600/101; 216/24; 216/97; 269/909

(57) ABSTRACT

A method of fabricating an optical fiber includes placing a distal end of an optical fiber in an etch-proof fiber-holder such that a tip portion of the optical fiber is covered by the fiber-holder, and an etching portion of the optical fiber adjacent to the tip portion is not covered by the fiber-holder. The optical fiber is exposed to an etching solution to change a profile of the etching portion, and the distal end of the optical fiber is removed from the fiber-holder.

### BACKGROUND OF THE INVENTION

1. Field of the Invention

The present invention relates to an optical fiber for an endoscope, which guides or directs light toward a distal end of the endoscope. In particular, it relates to a fabricating process for a scanning optical fiber that vibrates in resonance to scan a beam over a region of tissue.

2. Description of the Related Art

In an endoscope with a scanning optical fiber, a single mode optical fiber is provided in the endoscope, and is attached to a piezoelectric 2-D actuator so that the distal end of the fiber becomes a cantilever beam. The piezoelectric actuator 2-dimensionally vibrates the cantilevered distal end at a resonant frequency while modulating or amplifying amplitudes of the vibration, so that the tip portion of the optical fiber is driven in a spiral pattern. A plurality of photodetectors, which are provided around the distal end of the optical fiber, detect light reflected from a region of tissue, and a sequence of signals are transmitted from the photodetectors to a processor connected to the endoscope. The spiral scanning is repeatedly performed by a frame rate, so that a full-color movie-image is displayed on a monitor connected to the processor.

The resolution or definition and the field of view of an acquired image depend upon the resonant frequency and the maximum amplitude. In order to vibrate the optical fiber at a desired frequency, maximum amplitude, or desired resonant mode, the diameter of the optical fiber is reduced at a portion adjacent to the tip portion of the optical fiber. However, it can be difficult to precisely configure the end of an optical fiber in order to achieve a desired frequency, amplitude or resonant mode.

### SUMMARY OF THE INVENTION

Therefore, an object of the present invention is to provide an optical fiber fabricating process for producing precisely configured optical fibers, and apparatus for use in such a process. Another object of the present invention is to provide optical fibers with precisely configured distal ends, and endoscopes including such optical fibers.

According to one aspect of the present invention, there is provided a method for fabricating an optical fiber which includes placing a distal end of an optical fiber in an etch-proof fiber-holder such that a tip portion of the optical fiber is covered by the fiber-holder, and an etching portion of the optical fiber adjacent to the tip portion is not covered by the fiber-holder; exposing the optical fiber to an etching solution to change a profile of the etching portion; and removing the distal end of the optical fiber from the fiber-holder.

The method may include shifting the distal end of the optical fiber in a longitudinal direction while exposing the optical fiber to the etching solution. The etching solution may be provided in layers including a layer of etchant, a layer of etch-proof solution that is disposed below the etchant layer and is non-reactive with the etchant, and a layer of organic solvent that is disposed above the etchant layer and is non-reactive with the etchant. The etchant may include a hydrogen fluoride (HF) solution. The etch-proof solution may include fluorinated oil. The organic solvent may include iso-octane.

The method may include coating the distal end of the optical fiber with a metallic material before placing the distal end of the optical fiber in the etch-proof fiber-holder; and removing the metallic material coated on the etching portion of the optical fiber by exposing the optical fiber to a metal etchant before exposing the optical fiber to the etching solution. The method may include holding the distal end of the optical fiber in the fiber-holder with an adhesive. The fiber-holder may have an aperture through which the etching portion of the optical fiber is exposed to the exterior of the fiber-holder. The fiber-holder may be formed of silicon material. The optical fiber may be immersed in the etching solution so as to reduce a cross-sectional area of the etching portion relative to that of the tip portion.

The method may further include detecting a diameter and a length of the etching portion during exposure of the optical fiber to the etching solution. The detecting may include observing the etching portion with a microscope. Exposure to the etching solution may change the profile of the etching portion to a profile that allows vibration of the distal end of the optical fiber in a second resonant mode. The optical fiber may be a single-mode optical fiber.

The fiber-holder may include a pair of holding members, the distal end of the optical fiber being placed between the pair of holding members. Each of the holding members may have a supporting surface with a groove that supports the distal end of the optical fiber. Each groove may have a generally V-shaped cross-section. Each of the holding members may have an aperture through which the etching portion of the optical fiber is exposed to the exterior of the fiber-holder. A size of each aperture may correspond to the etching portion of the optical fiber. The holding members may be connected together such that the apertures are aligned. Each of the holding members may have a supporting surface with a groove that supports the distal end of the optical fiber, each of the grooves having aligned groove portions on opposite sides of the respective aperture.

According to another aspect of the present invention, there is provided an endoscope including an optical fiber fabricated by the method according to the above-noted aspect of the invention; and an actuator that vibrates a distal end of the optical fiber so as to scan light irradiated from the optical fiber over an area.

The actuator may be a piezoelectric actuator which vibrates the distal end of the optical fiber in resonance. The endoscope may include at least one photo-detector that receives light reflected from the area. The endoscope may include a plate located at a distal end of the endoscope, the actuator being mounted on the plate. A plurality of photo-detectors may be mounted on the plate concentrically around the actuator. The plate may include a hole through which the optical fiber extends, the photo-detectors being located concentrically around the hole.

According to another aspect of the present invention, there is provided a fiber-holder for holding an optical fiber during etching, the fiber-holder including a first etch-proof holding member having a first aperture and a first supporting surface that supports a distal end of an optical fiber; and a second etch-proof holding member having a second aperture and a second supporting surface that supports the distal end of the optical fiber, wherein the first holding member and the second holding member are configured to hold the distal end of the optical fiber therebetween.

The first holding member and the second holding member may be connected together such that the first aperture and the second aperture are aligned. A portion of the distal end of the optical fiber may extend across the first aperture and the second aperture so as to be exposed to the exterior of the fiber-holder. The first supporting surface may include a first groove configured to support the distal end of the optical fiber, and the second supporting surface may include a second groove configured to support the distal end of the optical fiber. The first groove may include aligned groove portions on opposite sides of the first aperture, and the second groove may include aligned groove portions on opposite sides of the second aperture, such that a portion of the distal end of the optical fiber extends across the first aperture and the second aperture so as to be exposed to the exterior of the fiber-holder.

### BRIEF DESCRIPTION OF THE DRAWINGS

The present invention is further described in the detail description which follows, in reference to the noted plurality of drawings, by way of non-limiting examples of preferred embodiments of the present invention, in which like characters represent like elements throughout the several views of the drawings, and wherein:

FIG. 1 is a block diagram of an endoscope system according to an embodiment of the invention;

FIG. 2 is a sectional view showing the distal end of the endoscope of FIG. 1;

FIG. 3 is a sectional view similar to FIG. 2 depicting a vibrating optical fiber;

FIG. 4 is a perspective view depicting a method for manufacturing a fiber-holder;

FIG. 5 is a perspective view depicting a process for fixing an optical fiber in a fiber-holder;

FIG. 6 is a sectional view depicting a fabrication process of the optical fiber before an etching process;

FIG. 7 is a sectional view depicting a fabrication process of the optical fiber after an etching process; and

FIG. 8 is a perspective view showing equipment for etching an optical fiber.

### DETAILED DESCRIPTION

The present invention will be described below with reference to the embodiments shown in the drawings.

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

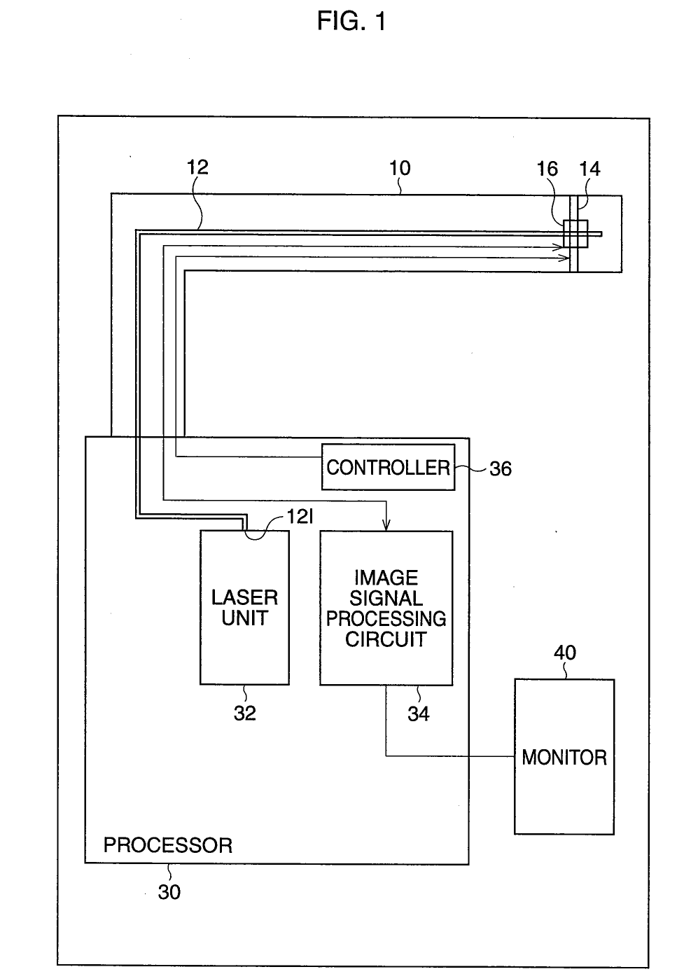

FIG. 1 is a block diagram of an endoscope system according to the present embodiment.

The endoscope system may include an endoscope 10, a processor 30, and a monitor 40. The endoscope 10 may be detachably connected to the processor 30, and a suitable optical fiber, such as a single-mode optical fiber 12, extends through the endoscope 10. A monitor 40 is connected to the processor 30.

A laser unit 32 is provided in the processor 30, and is configured to emit a laser beam. Irradiated light enters an incident surface 121 of the optical fiber 12. The optical fiber 12 is configured to guide or direct the light from the proximal end to the distal end 10A of the endoscope 10. Light passing through the optical fiber 12 exits from the tip portion of the endoscope 10, so that an observed portion or surface is illuminated. Light reflected off the observed portion or surface enters a plurality of photo-detectors 14, and image-pixel signals are successively read from the photo-detectors 14 to by an image signal processing circuit 34 in the processor 30. In the image signal processing circuit 34, the signals are subjected to various processes to generate image signals. The generated image signals are fed to the monitor 40, so that the object image is displayed on the monitor 40. A controller 36 controls a piezoelectric actuator 16 provided at the distal end 10A of the endoscope 10.

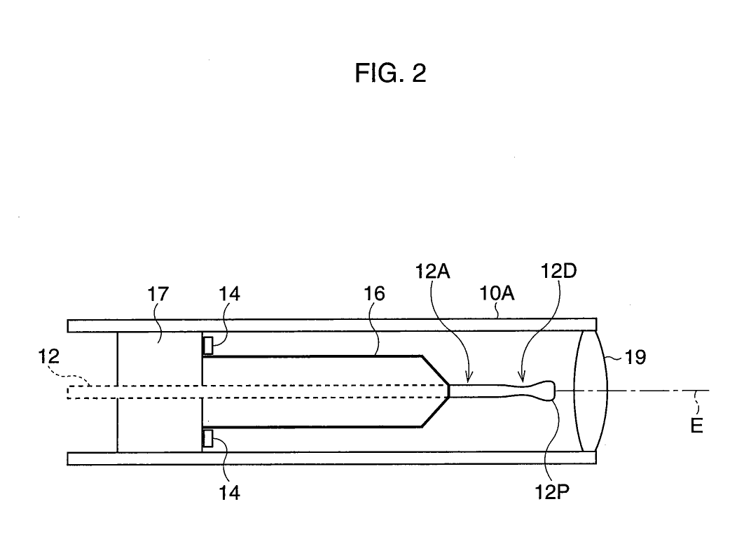

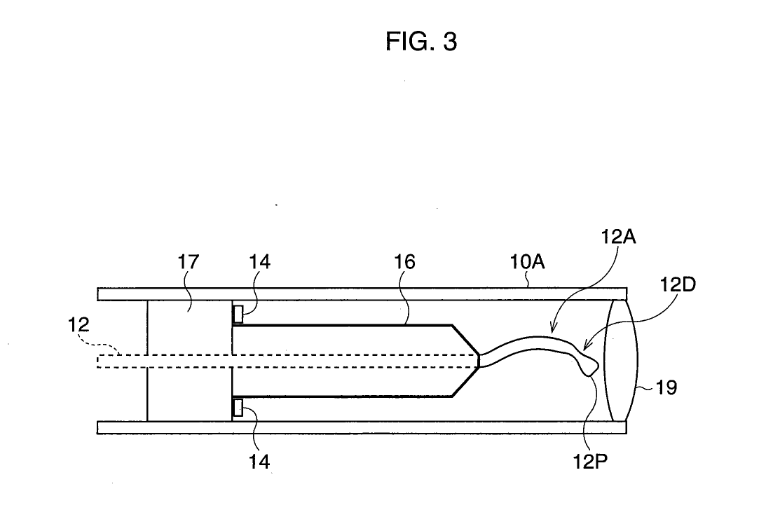

FIG. 2 is a view showing an inner construction of the distal end of the endoscope. FIG. 3 is a view depicting a vibrating optical fiber in the distal end of the endoscope.

In the distal end 10A of the endoscope 10, a 2-D piezoelectric actuator 16 is disposed and mounted to the fixed plate 17. The optical fiber 12 may extend through the actuator 16 and the distal end 12A of the optical fiber 12 may be held by the actuator 16. As shown in FIG. 2, the distal end 12A of the optical fiber extends along a line E in a stationary or rest condition. A lens 19, which may be disposed at the tip portion of the endoscope 10, is configured to pass and deflect light from the optical fiber 12 such that the light exits toward the tissue. The plurality of photo-diodes 14 may be arranged concentrically around the actuator 16, and mounted to the fixed plate 17 circumferentially, at constant intervals.

The distal end 12A of the optical fiber 12 may project from the actuator 16 so that the distal end 12A becomes a fixed-free cantilever. The actuator 16 may be of any suitable type, such as a tube-shaped piezoelectric actuator. Further, such a piezoelectric actuator 16 may be composed of any suitable type of piezoelectric materials, such as PZT. The piezoelectric actuator 16 may be deformed by an inverse piezoelectric effect, thereby two-dimensionally driving the distal end 12A of the optical fiber 12. Namely, the piezoelectric actuator 16 may vibrate the distal end 12A along two axes perpendicular to each other while modulating or amplifying amplitudes of the vibration, so as to scan the distal tip portion 12P in spiral patterns. Thus, light irradiated from the distal tip 12P via the lens 19 may be scanned over the observed portion in the spiral pattern(s).

Light reflected from the observed portion passes through the lens 19 and the plurality of photo-detectors 14 collects the reflected light. Consequently, signals corresponding to the detected light are read from the photo-detectors 14 in a time-series, and are fed to the image signal processing circuit 34 shown in FIG. 1. R, G, and B color filters are disposed, respectively, on the photo-diodes 14 such that the balance or ratio of the colors R, G, and B are usually equal. In the image signal processing circuit 34, color in each pixel may be detected from signals fed from the plurality of photo-diodes 14. For example, when the ratio of signals fed from photo-diodes with an R filter is larger than that fed from photo-diodes with the other (G, B) filters, the pixel color is set to reddish color.

The single-mode optical fiber 12 may be formed of any suitable material, such as a silica core and silica cladding. Further, a suitable covering may also be provided, for example a metallic or plastic covering, such as nylon sheet. The diameter D of the optical fiber 12 may be set to a range between a few hundred microns and a few millimeters. The optical fiber 12 has an etched-region 12D, in which the cross-sectional area or diameter is reduced. The tip portion 12P of the optical fiber 12 is not etched and remains at the diameter D. The etched-region may be configured by the fabricating process described below. Reducing the diameter at the etched-region 12D enables the distal end 12A of the optical fiber 12 to vibrate in the second resonance mode (See FIG. 3). By resonating in the second resonance mode, the field of view of an acquired image becomes broader as compared to the vibrating in the first resonance mode, and the high frame-rate may be maintained regardless of the short-length of the etched-region 12D.

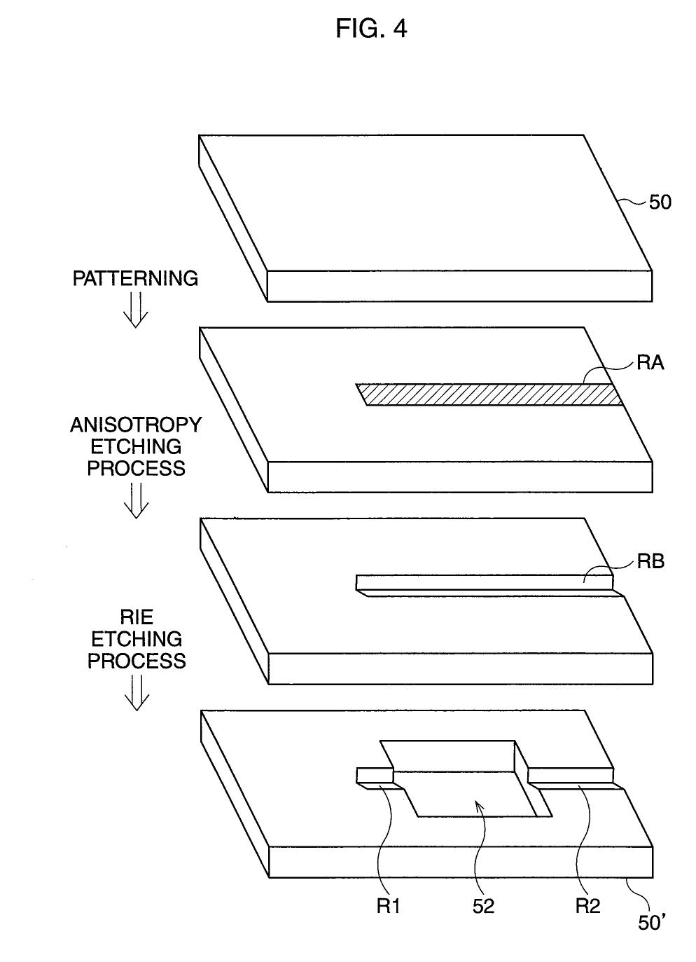

With reference to FIGS. 4 to 8, the fabrication process according to the present embodiment is explained.

FIG. 4 is a view showing a method for manufacturing a fiber-holder, which may be formed of any suitable material, such as silicon. Two rectangular silicon wafers are prepared and photosensitive material, such as photo-resist, is applied to the surface of the silicon wafer 50. In FIG. 4, one silicon wafer 50 is shown. The resist-covered silicon wafer 50 may be subjected to a pattern-forming or -writing process by using a beam writer that directly writes a pattern on a photosensitive substrate by a laser beam. A bar-like area RA extending along one direction may be patterned such that the size of the area RA corresponds to the size or diameter of the optical fiber 12, and the silicon wafer may be symmetrical with respect to the area RA.

The pattern-formed silicon wafer 50 may be subjected to a development process and an etching process, such as an anisotropy-etching process. In this manner, a groove RB configured to support the distal end 12A of the optical fiber 12 is formed on the area RA. The groove RB may be of any suitable shape, such as a V-shaped groove. After the etching process, the silicon wafer 50 may be subjected to a deep reactive ion etching (RIE) process from the back surface, so that an aperture 52 is formed through the wafer 50. The aperture 52 may be of any suitable shape, such as rectangular, and divides the groove RB into a groove R1 and a groove R2. The groove R1 is configured to support the tip portion 12P of the optical fiber 12, and the groove R2 is configured to support a remaining distal end portion 12A of the optical fiber 12. The dimensions of the aperture 52 may correspond to those of the etched-region 12D of the optical fiber 12.

A fiber holder can also be configured to hold a plurality of optical fibers 12 for batch fabrication. In this regard, a silicon wafer 50 may include a plurality of parallel grooves RB for holding a plurality of parallel optical fibers 12. Further, a plurality of optical fibers 12 may extend across a single aperture 52 of a silicon wafer 50. Alternatively, a separate aperture 52 may be provided in the silicon wafer 50 for each of the grooves RB and optical fibers 12.

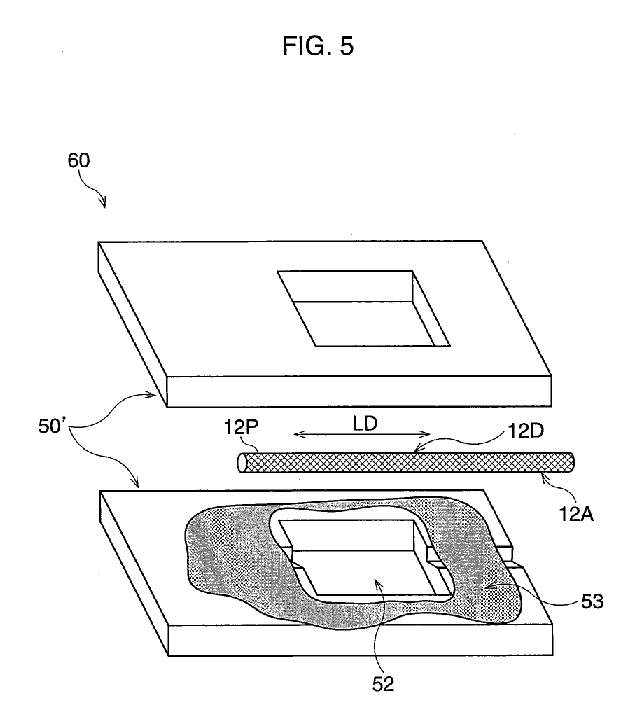

FIG. 5 is a view showing a process for fixing the optical fiber 12 in a fiber-holder 60.

The distal end 12A of the optical fiber 12 may be coated with any suitable material, such as a metal material, for example chromium, by any suitable process, such as vapor deposition or sputtering. Two silicon wafers 50′ manufactured by the process shown in FIG. 4 are prepared, and an adhesive or bonding agent 53 is applied around the aperture 52 of at least one of the silicon wafer 50′. The adhesive or bonding agent 53 may be of any suitable material, such as a photo-resist. The distal end 12A of the optical fiber 12 is then placed along the grooves R1 and R2 such that the tip portion 12P of the optical fiber 12 is on the groove R1, the remaining portion is on the groove R2, and an etching portion (to be formed into the etched-region 12D) extends across the aperture 52. The other silicon wafer 50′ may be connected to the adhesive-applied silicon wafer 50′. Thus, the distal end 12A of the optical fiber 12 is fastened or clamped between the two silicon wafers 50′, which form the fiber-holder 60. The etched-region 12D may have a length “LD” exposed to the outside of the fiber-holder 60 by the aperture 52, and the tip portion 12P is disposed in, and covered by, the impervious etch-proof fiber-holder 60.

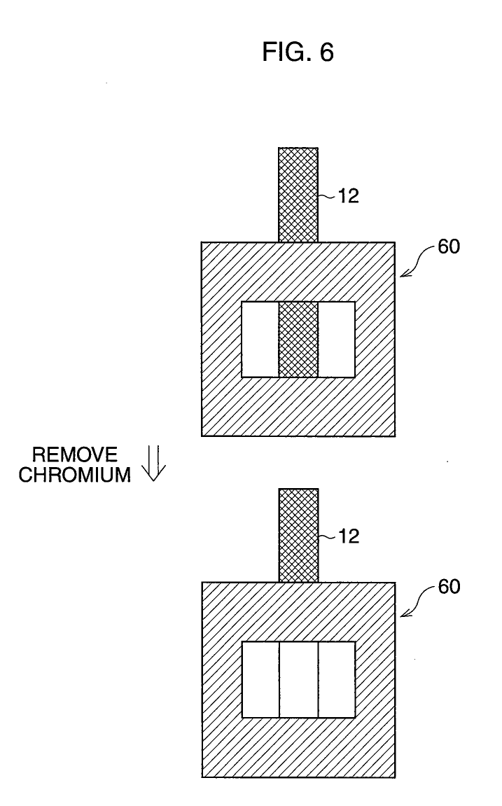

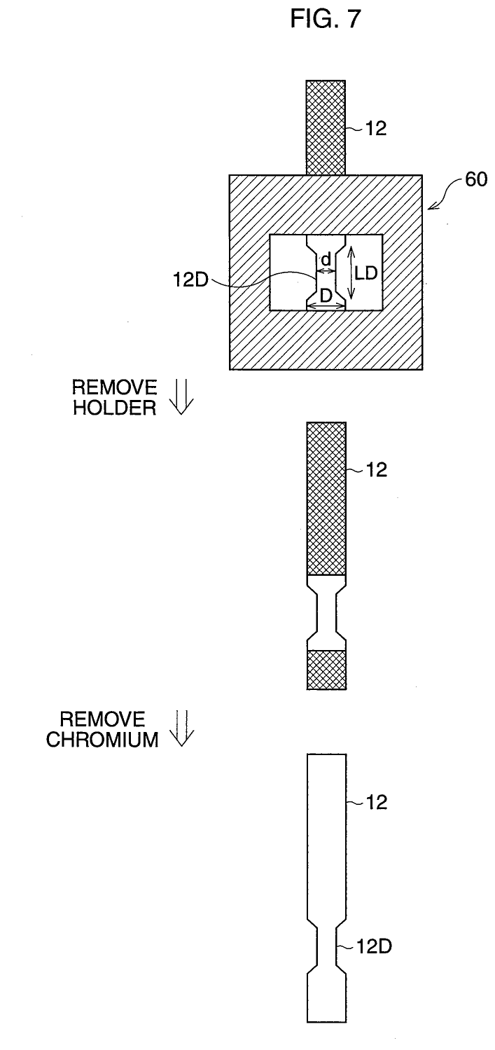

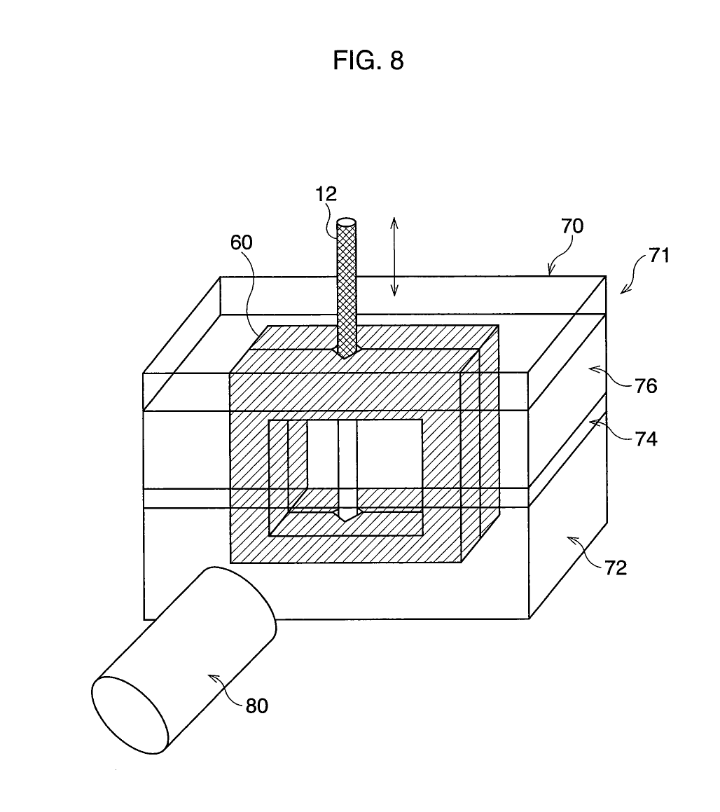

FIG. 6 is a view showing a fabrication process of the optical fiber before the etching process. FIG. 7 is a view showing a fabrication process of the optical fiber after the etching process. FIG. 8 is a view showing equipment for the etching process.

The optical fiber 12 fastened to the fiber-holder 60 may be exposed, such as by immersion, in a suitable metal etchant, such as a chromium etchant. In this manner the chromium coating the etching portion (to be formed into the etched-region 12D) is etched and removed from the etching portion of the optical fiber (see FIG. 6).

Subsequently, the optical fiber 12 fastened to the fiber-holder 60 may be exposed, such as by immersion, in a suitable etching solution 71, as shown in FIG. 8. One example of a suitable etching solution 71 contained in a vessel or chamber 70 includes three liquid layers of fluorinated oil 72, etchant 74, and organic solvent 76. The fluorinated oil 72 may be denser than the other liquids 74 and 76, and is immiscible and non-reactive with the etchant 74. The etchant 74 may be hydrogen fluoride (HF) solution, and include hydrofluoric acid. The organic solvent 76 including iso-octane is immiscible with the hydrofluoric acid and prevents any etching by the HF acid vapor, which is released by the hydrogen fluoride solution 74. However, any suitable etching solution may be used.

A suitable actuator, such as a linear actuator (not shown), may be connected to the optical fiber 12 to enable the optical fiber 12, and particularly the distal end 12A, to be shifted or reciprocated along its longitudinal axis during exposure to the etching solution.

The optical fiber 12 may be exposed (or immersed) in the vessel 70 such that part of the etching portion (to be formed into the etched-region 12D) is located in the etchant 74. Further, the optical fiber 12 may be gradually and intermittently moved along the longitudinal direction so as to form a required profile of the etched-region 12D, or the total of the distal end 12A. As shown in FIG. 8, a microscope 80 is arranged to detect or observe the profile of the etched-region 12D of the optical fiber 12 during the etching process. The operator measures the diameter and the length of the etched-region 12D by using the microscope 80, and continues moving the optical fiber 12 until the profile of the etched-region 12D becomes the desired profile. The diameter “d” and the length “LD” of the etched-region 12D (see FIG. 7) are determined in accordance with the desired resonant frequency, amplitudes of the vibration, vibratory node position, and so on. The diameter “d” and the length “LD” may be determined such that the optical fiber 12 vibrates in a second resonant mode.

After the etching process, the optical fiber 12 may be detached from the actuator. Then the optical fiber 12 may be detached from the holder 60 in a suitable manner, such as by using resist stripper or sulfuric acid. To remove the chromium covering the optical fiber 12, the optical fiber 12 may be exposed, such as by immersion in chromium etchant. Thus, the fabricated optical fiber 12 may be completed, as shown in FIG. 7.

In this way, the distal end 12A of the optical fiber 12 is gripped between the fiber-holder 60, which is composed of the two silicon wafers 50′ and has the aperture 52, and is fastened or clamped to the fiber-holder 60 by using the adhesive agent 53. The optical fiber 12 is immersed into the etching solution 71 with the etchant 74. The fiber-holder 60 functions as a weight or load during the etching process, and the fiber-holder 60 has symmetry or balance with respect to the held optical fiber 12. Therefore, the optical fiber 12 is stable while the distal end 12A is submerged in the etching solution 71, and the distal end 12A including the tip portion 12P does not vibrate or swing (even if the etchant solution 71 is disturbed during immersion of the optical fiber 12 in the etching solution, such as by moving the optical fiber 12 along the longitudinal direction). Thus, the operator can precisely or correctly measure the diameter “d” and the length “LD” of the etched-region 12D and the profile of the etched-region 12D during the etching process. Consequently, the distal end 12A of the optical fiber 12 is finely and precisely configured so as to realize the vibration in the second resonant mode.

Further, since the etched-region 12D is exposed through the aperture 52 to the exterior of the fiber-holder 60, and the tip portion 12P is sealed within the fiber-holder 60, the cross-sectional area of the optical fiber 12 is reduced at only the etched-region 12D, and the other portion, especially the cross-sectional area of the tip portion 12P, is not reduced while submerging the optical fiber 12 in the etching solution and moving the optical fiber 12 along the longitudinal direction.

As for the etching solution 71, another suitable etchant can be used instead of the HF acid solution. Further, another suitable etch-proof solvent can be used instead of fluorinated oil, and another suitable organic solvent can be used instead of iso-octane solvent. Further, the etching solution 71 may consist of only etchant, or any other suitable combination of solutions. Another suitable metallic or plastic material can be used to coat the optical fiber, instead of chromium.

Optionally, the position of the optical fiber 12 in the vessel 70 may be fixed throughout the etching process. In this case, the depth of the etchant layer 74 may be varied or adjusted to control the etching process. An optional profile of the optical fiber can be realized by the etching process according to the present embodiment. Therefore, the profile of the etched-region that allows the optical fiber to vibrate in first resonant mode or multi-resonant mode (for example, third resonant mode) can be determined. Further, another optical fiber, such as multi-mode optical fiber, can be used in the etching process.

Optionally, the optical fiber can be fastened to the fiber-holder using another method instead of adhesion. Optionally, a fiber-holder that is not etched by the etching solution, and that is capable of sealing the optical fiber, can be utilized, instead of the fiber-holder 60 described above. For example, a one-piece fiber-holder having a long and narrow hole can be prepared in advance, and the optical fiber can be installed into the hole and fasten to the fiber-holder. The configuration of the fiber-holder may be defined such that the etched-region is exposed outside of the fiber-holder. Also, a fiber-holder composed of another material, such as sapphire, which is impervious and resist-durable to the etchant solution, can be used, instead a fiber-holder composed of silicon wafer. In the case of sapphire, the fiber-holder can be manufactured by a sandblasting. The present invention can also be implemented in other applications other than endoscopy, such as microscopy and confocal microscopy. Finally, it will be understood by those skilled in the arts that the foregoing description is of preferred embodiments of the device, and that various changes and modifications may be made to the present invention without departing from the spirit and scope thereof.

It is further noted that the foregoing examples have been provided merely for the purpose of explanation and are in no way to be construed as limiting of the present invention. While the present invention has been described with reference to a preferred embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The above disclosed subject matter is to be considered illustrative, and not restrictive, and the appended claims are intended to cover all such modifications, enhancements, and other embodiments which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

Although the invention has been described with reference to several exemplary embodiments, it is understood that the words that have been used are words of description and illustration, rather than words of limitation. As the present invention may be embodied in several forms without departing from the spirit or essential characteristics thereof, it should also be understood that the above-described embodiments are not limited by any of the details of the foregoing description, unless otherwise specified. Rather, the above-described embodiments should be construed broadly within the spirit and scope of the present invention as defined in the appended claims. Therefore, changes may be made within the metes and bounds of the appended claims, as presently stated and as amended, without departing from the scope and spirit of the invention in its aspects.

What is claimed is:

1. A method for fabricating an optical fiber comprising:
placing a distal end of an optical fiber in an etch-proof fiber-holder such that a tip portion of the optical fiber is covered by the fiber-holder, and an etching portion of the optical fiber adjacent to the tip portion is not covered by the fiber-holder;
exposing the optical fiber to an etching solution to change a profile of the etching portion; and
removing the distal end of the optical fiber from the fiber-holder.

2. The method of claim 1, further comprising shifting the distal end of the optical fiber in a longitudinal direction while exposing the optical fiber to the etching solution.

3. The method of claim 1, wherein the etching solution is provided in layers including a layer of etchant, a layer of etch-proof solution that is disposed below the etchant layer and is non-reactive with the etchant, and a layer of organic solvent that is disposed above the etchant layer and is non-reactive with the etchant.

4. The method of claim 3,wherein the etchant includes a hydrogen fluoride (HF) solution.

5. The method of claim 3, wherein the etch-proof solution includes fluorinated oil.

6. The method of claim 3, wherein the organic solvent includes iso-octane.

7. The method of claim 1, further comprising:
coating the distal end of the optical fiber with a metallic material before placing the distal end of the optical fiber in the etch-proof fiber-holder; and
removing the metallic material coated on the etching portion of the optical fiber by exposing the optical fiber to a metal etchant before exposing the optical fiber to the etching solution.

8. The method of claim 1, further comprising holding the distal end of the optical fiber in the fiber-holder with an adhesive.

9. The method of claim 1, wherein the fiber-holder has an aperture through which the etching portion of the optical fiber is exposed to the exterior of the fiber-holder.

10. The method of claim 1, wherein the fiber-holder is formed of silicon material.

11. The method of claim 1, wherein the optical fiber is immersed in the etching solution so as to reduce a cross-sectional area of the etching portion relative to that of the tip portion.

12. The method of claim 1, further comprising detecting a diameter and a length of the etching portion during exposure of the optical fiber to the etching solution.

13. The method of claim 12, wherein the detecting includes observing the etching portion with a microscope.

14. The method of claim 1, wherein exposure to the etching solution changes the profile of the etching portion to a profile that allows vibration of the distal end of the optical fiber in a second resonant mode.

15. The method of claim 1, wherein the optical fiber is a single-mode optical fiber.

16. The method of claim 1, wherein the fiber-holder includes a pair of holding members, and the distal end of the optical fiber is placed between the pair of holding members.

17. The method of claim 16, wherein each of the holding members has a supporting surface with a groove that supports the distal end of the optical fiber.

18. The method of claim 17, wherein each groove has a generally V-shaped cross-section.

19. The method of claim 16, wherein each of the holding members has an aperture through which the etching portion of the optical fiber is exposed to the exterior of the fiber-holder.

20. The method of claim 19, wherein a size of each aperture corresponds to the etching portion of the optical fiber.

21. The method of claim 19, wherein the holding members are connected together such that the apertures are aligned.

22. The method of claim 19, wherein each of the holding members has a supporting surface with a groove that supports the distal end of the optical fiber, each of the grooves having aligned groove portions on opposite sides of the respective aperture.

23. An endoscope comprising:
an optical fiber fabricated by the method according to claim 1; and
an actuator that vibrates a distal end of the optical fiber so as to scan light irradiated from the optical fiber over an area.

24. The endoscope according to claim 23, wherein the actuator is a piezoelectric actuator which vibrates the distal end of the optical fiber in resonance.

25. The endoscope according to claim 23, further comprising at least one photo-detector that receives light reflected from the area.

26. The endoscope according to claim 23, further comprising a plate located at a distal end of the endoscope, the actuator being mounted on the plate.

27. The endoscope according to claim 26, further comprising a plurality of photo-detectors mounted on the plate concentrically around the actuator.

28. The endoscope according to claim 27, wherein the plate includes a hole through which the optical fiber extends, and the photo-detectors are located concentrically around the hole.

29. A fiber-holder for holding an optical fiber during etching, the fiber-holder comprising:
a first etch-proof holding member having a first aperture and a first supporting surface that supports a distal end of an optical fiber; and
a second etch-proof holding member having a second aperture and a second supporting surface that supports the distal end of the optical fiber,
wherein the first holding member and the second holding member are configured to hold the distal end of the optical fiber therebetween.

30. The fiber-holder according to claim 29, wherein the first holding member and the second holding member are connected together such that the first aperture and the second aperture are aligned.

31. The fiber-holder according to claim 30, wherein a portion of the distal end of the optical fiber extends across the first aperture and the second aperture so as to be exposed to the exterior of the fiber-holder.

32. The fiber-holder according to claim 29, wherein the first supporting surface includes a first groove configured to support the distal end of the optical fiber, and the second supporting surface includes a second groove configured to support the distal end of the optical fiber.

33. The fiber-holder according to claim 32, wherein the first groove includes aligned groove portions on opposite sides of the first aperture, and the second groove includes aligned groove portions on opposite sides of the second aperture, such that a portion of the distal end of the optical fiber extends across the first aperture and the second aperture so as to be exposed to the exterior of the fiber-holder.
